(19) United States
(12) Patent Application Publication
(10) Pub. No.: US 2017/0029482 A1
(43) Pub. Date: Feb. 2, 2017

(54) NOVEL PURIFICATION PROCESS OF GONADOTROPIN

(71) Applicant: CADILA HEALTHCARE LIMITED, Ahmedabad, Gujarat (IN)

(72) Inventors: Sanjeev Kumar Mendiratta, Ahmedabad, Gujarat (IN); Sanjay Bandyopadhyay, Ahmedabad, Gujarat (IN); Avanish K. Singh, Ahmedabad, Gujarat (IN); Mithra S. Reddy, Ahmedabad, Gujarat (IN)

(21) Appl. No.: 15/303,888

(22) PCT Filed: Apr. 17, 2015
(86) PCT No.: PCT/IN2015/000175

§ 371 Date: Oct. 13, 2016

(30) Foreign Application Priority Data
Apr. 18, 2014 (IN) 1398/MUM/2014

(51) Int. Cl. C07K 14/59

(52) U.S. Cl. CPC C07K 14/59

(57) ABSTRACT

The present invention provides an improved method for the purification of desired gonadotropin from a crude mixture containing at least one contaminating protein. The process of purification of the desired gonadotropin according to the present invention comprises use of an affinity chromatography as the first column purification step, prior to use of any column chromatography steps for further purification. Such purification process may further include ion exchange and/or hydrophobic interaction chromatography step to obtain substantially purified gonadotropin protein with desired isoforms profile.

### FIELD OF THE INVENTION

The present invention provides an improved method for the purification of desired gonadotropin from a crude mixture containing at least one contaminating protein. The process of purification of the desired gonadotropin according to the present invention comprises use of an affinity chromatography as the first column purification step, prior to use of any column chromatography steps for further purification. Such purification process may further include ion exchange and/or hydrophobic interaction chromatography step to obtain substantially purified gonadotropin protein with desired isoforms profile.

### BACKGROUND OF THE INVENTION

Follicle Stimulating Hormone is a heterodimeric glycoprotein comprising of alpha (92 amino acids) and beta (111 amino acids) subunits. Glycosylation occurs on specific sites of the both the alpha and beta subunits Follicle Stimulating Hormone controls ovarian follicular growth, in female, and exhibits important role in inducing spermatogenesis, in men. Follicle Stimulating Hormone is indicated for the following therapeutic uses
Anovulation in women
Controlled ovarian hyper stimulation to induce the development of multiple follicles in women for in-vitro fertilization (IVF)/Embryo transfer (ET)
Follicle Stimulating Hormone in combination with LH is recommended for the stimulation of follicular development in women
In male, with hypogonadotropic hypogonadism with concomitant hCG therapy.

The inventors of the present invention have indigenously developed the recombinant r-hFSH or Follitropin, by r-DNA technology using the genetically engineered CHO cells as host system.

The present invention is related to purification of gonadotropins. There are several purification processes known in prior art for purification of gonadotropins. Such purification processes include use of high performance liquid chromatography (HPLC) which is expensive and requires a large amount of organic solvent during operation (e.g. patent document WO2006/051070). The high cost of the instrument and requirement of large excess of organic solvents are the major limitations in the case of purification of gonadotropin(s) by HPLC at industry scale.

WO2007/065918 discloses method for purifying FSH or a FSH mutant comprising the steps of subjecting a liquid containing said FSH or a FSH mutant to: (1) a dye affinity chromatography; (2) a weak anion exchange chromatography (3) a hydrophobic interaction chromatography; and (4) a strong anion exchange chromatography; which may be carried out in any order. It includes an optional step of capture step before the first step of dye affinity chromatography purification step as step (0).

Dye affinity chromatography is a protein purification procedure based on the affinity of immobilized dyes for the binding sites on many proteins. This chromatography technique is non-specific. An immobilized dye can bind to glycosylated protein molecule, nonspecifically. Another drawback of this purification technique is that there may be a chance of co-elution of other similar type of proteins present in the crude mixture along with the protein of interest. Moreover, there is also possibility of co-elution of dye molecule or its parts along with desired parts. So, it does not provide satisfactory level of purity of desired protein. The main disadvantage of these synthetic dyes is that the selection process for a particular biomolecule is empirical and requires extensive screening processes during method development. While, present invention does not include dye affinity chromatography step. Thus, in the purification process described here avoids chemical contamination of dyes or modified dyes.

WO 2005/063811 discloses a method for purifying recombinant human FSH or an FSH variant, comprising the steps of ion exchange chromatography; immobilized metal ion chromatography; and hydrophobic interaction chromatography (HIC) which may be carried out in any order.

The process described in the present invention for purification of gonadotropin does not include use of HPLC. Thus, the present invention discloses a simple, cost-effective, highly scalable, industrially viable and environmentally favorable process of purification to obtain highly purified gonadotropins. The process of purification disclosed in the present invention can also be used for purifying mixture of gonadotropins from a crude mixture.

Objective of this invention is to provide a new, advantageous method for purifying recombinant FSH or its functional variants. In the present invention, a novel process for purification of the recombinant human follicle stimulating hormone has been disclosed, in which no HPLC process step is used.

### SUMMARY OF THE INVENTION

The present invention provides a method for purifying gonadotropins from crude mixture. Crude mixture may include contaminating proteins, endogenous proteins, product related substances and other impurities in addition to the desired protein.

In one aspect, the present invention provides a process of purification of gonadotropins from a crude mixture comprising a series of chromatography steps which does not include HPLC.

In one of the embodiments, the present invention provides a purification process of cell culture derived gonadotropins from a crude mixture by using an affinity column chromatography, first to capture, and then elute the protein from the column with high level of purity. Crude mixture may include host-cell derived contaminating proteins, product-related substances and other impurities in addition to that of the protein of interest.

The present invention also demonstrates the removal of majority of the host cell contaminating proteins by affinity chromatography while eluting the protein of interest out of the column at neutral buffer pH condition or under acidic pH condition with maximum recovery.

In one of the embodiments, the present invention also demonstrates that the molecular integrity of the desired gonadotropin protein after elution from affinity column, under neutral or acidic pH conditions remain unaltered for at least about 24 hours, as assessed by analytical HP-SEC.

In one of the embodiments, the present invention also provides purification of gonadotropins with desired isoforms in binding mode through an anion exchange column chromatography.

In another embodiment, the present invention provides the removal of residual process-related and product-related impurities from the desired protein fraction by using a hydrophobic interaction column chromatography in bind-elute mode. Elution of the desired protein is performed at lower conductance either in a linear fashion or in a step-wise manner.

In a preferred embodiment, purification of the desired gonadotropin derived from crude mixture is carried out as per the following steps:
1. Affinity chromatography
2. Anion exchange column chromatography, followed by other suitable purification techniques which is available in the knowledge of the person skilled in the art and which does not include HPLC.

In another embodiment, purification of the desired gonadotropin derived from cell culture is carried out as per the following purification steps:
1. Affinity chromatography
2. Anion exchange column chromatography
3. Hydrophobic interaction chromatography

The hydrophobic interaction chromatography step can be performed in any order after the affinity chromatography steps. The process of purification described in the present application can be further carried out by any purification technique which is available in the knowledge of the person skilled in the art and which does not include HPLC.

Such purification techniques include diafiltration, any column chromatography, nanofiltration or any other known purification technique.

The abbreviations used in the present description are defined below:
Affinity Matrix: Affinity column purification
AEX: Anion exchange column chromatography
DF: Diafiltration
HIC: Hydrophobic interaction column chromatography
HP-SEC: High performance-size exclusion chromatography
HPL: High Performance Liquid Chromatography
u-HCG: Urinary HCG
u-FSH: Urinary FSH
MWCO: Molecular weight cut-off
NaCl: Sodium chloride
UF: Ultrafiltration
WFI: Water for Injection

### BRIEF DESCRIPTION OF THE FIGURES

FIG. 1 illustrates elution profile of r-hFSH from crude mixture by affinity column chromatography step employed in the purification process.

FIG. 2 illustrates the polypeptide profile of affinity column eluted r-hFSH by non-reducing SDS-PAGE.

FIG. 3 illustrates the elution profile of r-hFSH by AEX column chromatography step employed in the purification process.

FIG. 4 illustrates the purity of anion-exchange column-purified r-hFSH by HP-SEC. The figure shows single peak purity of r-hFSH.

FIG. 5 illustrates the elution chromatography profile of r-hFSH by HIC chromatography step employed in the purification process.

FIG. 6 illustrates the purity of HIC-purified r-hFSH by analytical HP-SEC. The figure shows single peak purity of r-hFSH.

FIG. 7 illustrates the purity of the r-hFSH Drug Substance by HP-SEC.

FIG. 8 illustrates elution profile of u-HCG from crude mixture by affinity column chromatography step employed in the purification process.

FIG. 9 illustrates the elution profile of u-HCG by AEX column chromatography step employed in the purification process.

FIG. 10 illustrates the polypeptide profile of u-HCG by non-reducing SDS-PAGE.

FIG. 11 illustrates the polypeptide profile by SDS-PAGE of the purified u-FSH.

FIG. 12 illustrates the purity of u-FSH by HP-SEC.

### DETAILED DESCRIPTION OF INVENTION

The present invention provides a novel purification process for the desired gonadotropin preferably FSH or its functional variants.

In one of the embodiments, the present invention provides a purification process of gonadotropin(s) from a crude mixture comprising using first an affinity chromatography followed by the use of other column chromatography steps which does not include HPLC. Crude mixture may include contaminating proteins, endogenous proteins, product related substances and other impurities in addition to the desired protein.

In one of the embodiments, the present invention provides a novel process for purification of gonadotropin(s) comprising use of Affinity and ion exchange chromatography steps. Ion exchange chromatography can be anion exchange column chromatography or cation exchange column chromatography.

In one of the embodiments, column matrix for affinity chromatography step is selected from FSH-specific and gonadotropins-specific affinity matrix. In another embodiment, the column matrix for anion exchange chromatography step is selected from DEAE sepharose, Mono Q and Q sepharose XL, preferably Q sepharose.

In a preferred embodiment, the purification process of gonadotropin(s) includes the following chromatographic steps:
1. Affinity chromatography
2. Anion exchange or cation exchange column chromatography
3. HIC chromatography

Such steps of column chromatography can be carried out in any order.

In another embodiment, the present invention provides the removal of residual process-related and product-related impurities from the desired protein fraction by using a hydrophobic interaction column chromatography in bind-elute mode. Elution of the desired protein is performed with down-the-gradient salt concentration in the form of a major peak.

In a further embodiment, the column matrix for hydrophobic interaction chromatography is selected from phenyl sepharose, butyl sepharose, octyl sepharose, preferably, phenyl sepharose.

In furthermore embodiment, the salt for elution of the desired protein at hydrophobic interaction chromatography step is selected from ammonium sulphate, sodium chloride, ammonium chloride and sodium sulphate preferably, ammonium sulphate.

In a more preferred embodiment, the purification of gonadotropin(s) from crude mixture is carried out as per the following steps:
Step 1: Cell separation and reconditioning
Step 2: Affinity column chromatography
Step 3: Viral inactivation
Step 4: Ultrafiltration-diafiltration and reconditioning (UF/DF)
Step 5: Anion Exchange column Chromatography (AEX)
Step 6: Reconditioning
Step 7: Hydrophobic interaction column chromatography (HIC)
Step 8: Ultrafiltration-diafiltration
Step 9: Virus clearance by nano-filtration
Step 10: Microfiltration
Step 11: Storage under frozen condition

In another embodiment, purification of the desired gonadotropin derived from crude mixture can be carried out without employing HIC chromatography steps.

In a further embodiment, the diafiltration medium is selected from water, Tris-Cl buffer, citrate buffer, phosphate buffer, succinate buffer, acetate buffer and combination thereof.

In a preferred embodiment, the gonadotropin is selected from follicle stimulating hormone (FSH), luteinizing hormone (LH), human chorionic gonadotropin (HCG) and suitable combinations thereof.

In a more preferred embodiment, the gonadotropin is selected from r-hFSH, u-FSH, r-hLH, u-LH, r-hHCG and u-HCG.

The column chromatography steps according to the present invention are described in further details below:

### I) Affinity Column Chromatography:

The clarified supernatant after reconditioning is passed through a gonadotropin-specific affinity column matrix to capture the desired gonadotropin, selectively, from a crude mixture. Prior to elution of the desired protein, the affinity matrix undergoes an intermediate column wash. The desired protein is eluted from the column at around neutral pH.

### II) Anion Exchange Column Chromatography

After diafiltration, solution containing recombinant follicle stimulating hormone is loaded on to an anion exchange column for further purification of the desired protein with desired isoforms profile. This column step is carried out in bind-elute mode and is performed mainly for the removal of undesired isoforms of recombinant follicle stimulating hormone, while isolating the said protein with desired isoforms. Protein is loaded on to the column at about pH 8.0 to bind to the matrix. Column matrix is washed with the same equilibration buffer to remove the unbound contaminants. Following the equilibration buffer wash, a second wash is performed with a buffer of pH lower than the initial equilibration buffer pH. Subsequently, a third wash is performed at acidic pH in the presence of NaCl. Column is re-equilibrated with the equilibration buffer and elution of the desired protein is carried out with an increase in conductance. For carrying out anion exchange chromatography according to the present invention, other anion exchangers which also can be used can be selected from DEAE sepharose, Mono Q, Q sepharose XL, and the like. Anion exchanger Q sepharose has been used in the present invention.

### III) Hydrophobic Interaction Column Chromatography:

Purification of the desired gonadotropin protein from a mixture containing at least one undesired contaminant is conducted by hydrophobic interaction column chromatography in bind-elute mode. After completion of protein-loading on to the column, the desired gonadotropin protein is eluted from the column with down-the-gradient salt concentration i.e. with decreased conductivity compared to that of the equilibration buffer conductivity. Elution of the desired gonadotropin protein takes place in the form of a single peak. The eluted protein is collected in fractions and the fractions containing the desired level of purity are pooled together. For carrying out hydrophobic interaction column chromatography according to the present invention, HIC resins, like Phenyl sepharose, Butyl sepharose 4 FF, Octyl sepharose etc. can be used.

### Analytical Technique Used in the Present Invention

HP-SEC: Analytical size-exclusion chromatography (HP-SEC) is performed by using a TSK-3000 column equilibrated with sodium phosphate buffer of pH 6.7 containing sodium sulphate. Protein is eluted in an isocratic-mode at 0.5 mL/min.

The steps of purification according to the present invention are described in further details below:

### EXAMPLES

Here, the present invention is illustrated with the following non-limiting examples which should not be interpreted as limiting the scope of the invention in any way:

### Example 1

### Purification of Recombinant FSH

### Step 1: Cell Separation and Reconditioning

After harvesting the batch, cells are separated from the culture broth, first by centrifugation followed by depth filtration in order to obtain clear supernatant containing the protein of interest along with other soluble contaminants. Centrifugation is carried out at about 10,000 g×30 minutes. Depth filtration is performed by using a 0.45→0.22 μm membrane for further clarification. The clarified supernatant is reconditioned to tune up with the next affinity column equilibration buffer condition e.g. pH and conductance. This step is not required when gonadotropin obtained from urine will be purified.

### Step 2: Affinity Column Chromatography

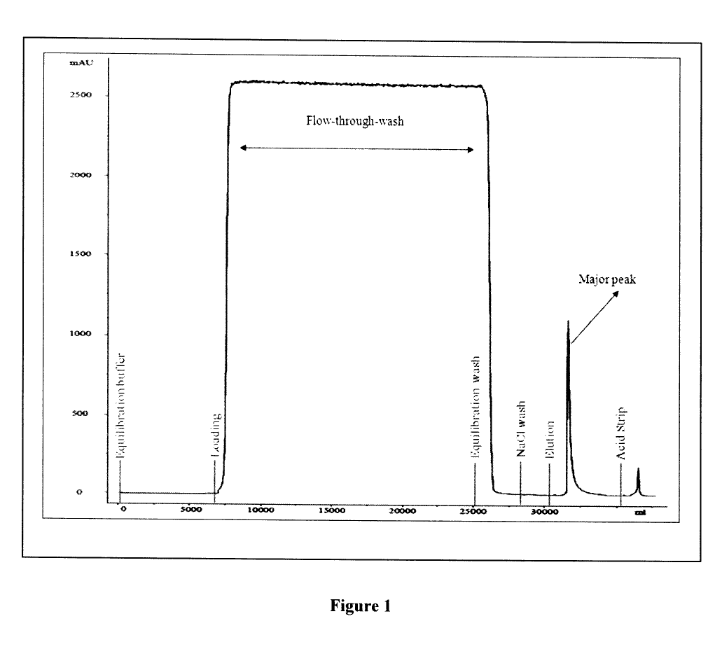

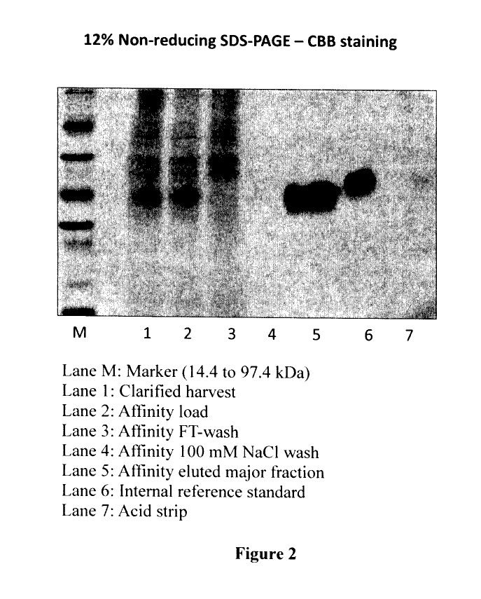

The clarified supernatant after reconditioning is passed through an affinity column matrix to capture the desired protein, selectively. Prior to elution of the desired protein, the affinity matrix undergoes an intermediate column wash. The desired protein is eluted from the column at around neutral pH. The column chromatography profile is shown in FIG. 1. The affinity-purified protein shows single band purity in gel, when analyzed by SDS-PAGE as shown in FIG. 2.

### Step 3: Ultrafiltration-Diafiltration and Reconditioning

The affinity column-eluted protein is reconditioned by UF/DF using 10 kDa MWCO membrane filter against low ionic strength Tris-Cl buffer of pH 7.0 in order to match to the next column (Q column) step equilibration buffer conditions (e.g. pH and conductance). Diafiltered protein solution is passed through a 0.22 μm filter, prior to loading on to the Q-column.

### Step 4: Viral Inactivation

Diafiltered protein solution is incubated at the same pH condition in the presence of solvent/detergent or detergent for about 4-6 hours, under room temperature condition with constant stirring for viral inactivation.

### Step 5: Anion Exchange Column Chromatography (AEX)

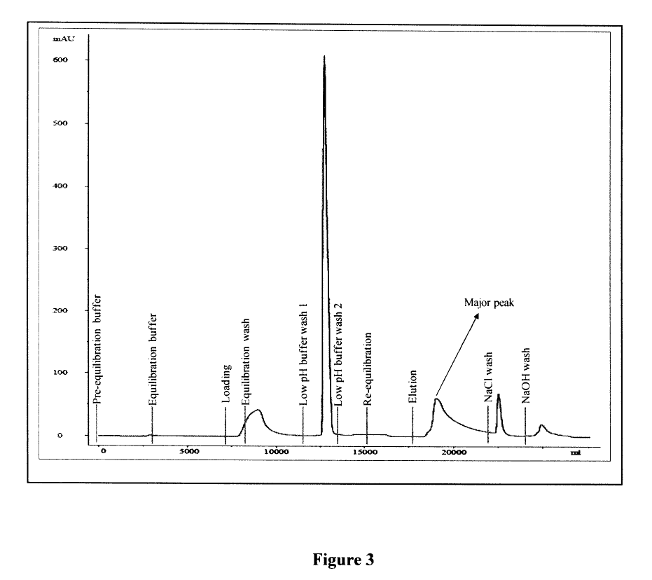

After diafiltration, solution containing recombinant follicle stimulating hormone is loaded on to an anion exchange column for further purification of the desired protein with desired isoforms profile. This column step is carried out in bind-elute mode and is performed mainly for the removal of undesired isoforms of recombinant follicle stimulating hormone, while isolating the said protein with desired isoforms. The column chromatography profile is shown in FIG. 3. Protein is loaded on to the column at about pH 8.0 to bind to the matrix. Column matrix is washed with the same equilibration buffer to remove the unbound contaminants. Following the equilibration buffer wash, a second wash is performed with a buffer of pH lower than the initial equilibration buffer pH. Subsequently, a third wash is performed at acidic pH in the presence of NaCl. Column is re-equilibrated with the equilibration buffer and elution of the desired protein is carried out with an increase in conductance.

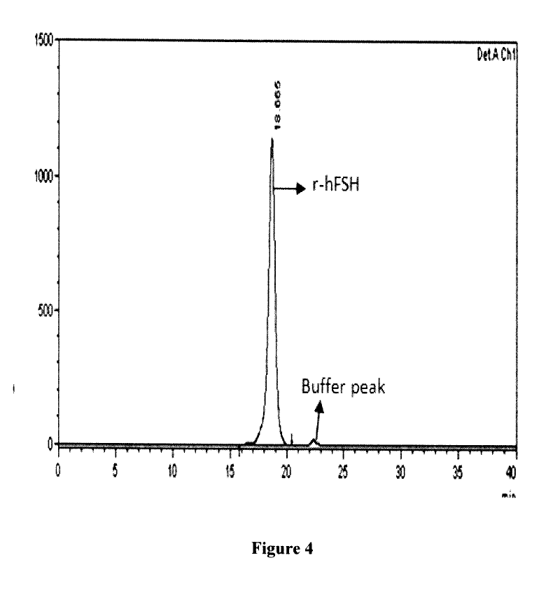

After the Q-column step, purity of the desired recombinant FSH protein is observed to be more than 98%, as assessed by HP-SEC shown in FIG. 4.

### Step 6: Reconditioning

Prior to loading on to the HIC column, the diafiltered protein solution is mixed with concentrated sodium chloride solution to tune-up, further, with the HIC column equilibration condition and passed through a 0.22 μm membrane filter.

### Step 7: Hydrophobic Interaction Column Chromatography (HIC)

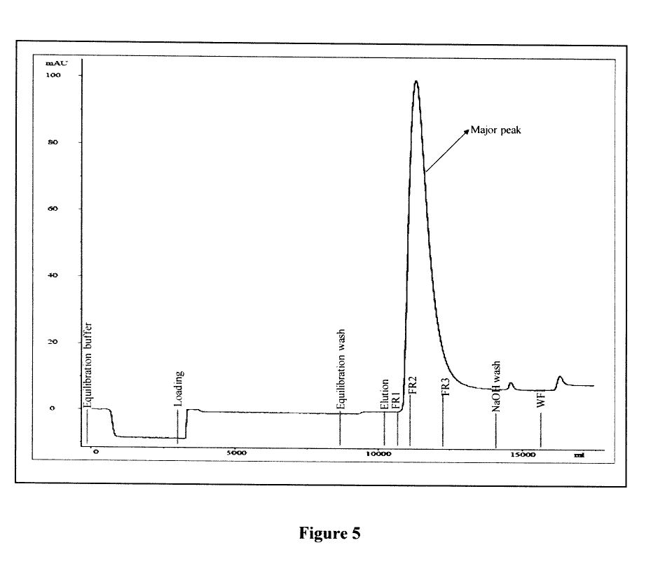

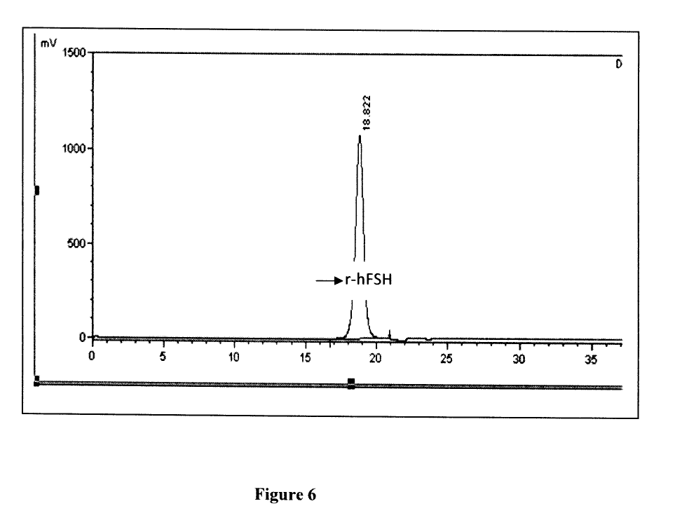

After reconditioning, the protein solution containing the desired protein is passed through a hydrophobic interaction chromatography matrix for further purification in bind-elute mode. Following binding to the column matrix, protein was eluted at lower conductance either in a linear fashion or in a step-wise manner. The column chromatography profile is shown in FIG. 5. The major eluted peak containing recombinant follicle stimulating hormone is collected for further processing. After the third column step, more than 99% purity of the desired recombinant FSH is achieved, as assessed by HP-SEC shown in FIG. 6.

### Step 8: Ultrafiltration-Diafiltration

After the third column step, solution containing recombinant follicle stimulating hormone undergoes an ultrafiltration-diafiltration step for buffer exchange, under room temperature conditions.

### Step 9: Nanofiltration

After the buffer exchange step, the recombinant follicle stimulating hormone undergoes a nanofiltration step for virus clearance. No significant loss of protein or aggregation is observed during and after the nanofiltration step, as assessed by HP-SEC. After nanofiltration, purity of recombinant follicle stimulating hormone is observed to be more than 99%.

Step 10: Microfiltration

Finally, the purified recombinant follicle stimulating hormone solution is passed through a 0.22 μm membrane filter, aseptically, and is stored either in the liquid form under cold condition (for short-term storage) or under frozen condition for long-term storage at a concentration between 0.2 mg/mL and 2.5 mg/mL.

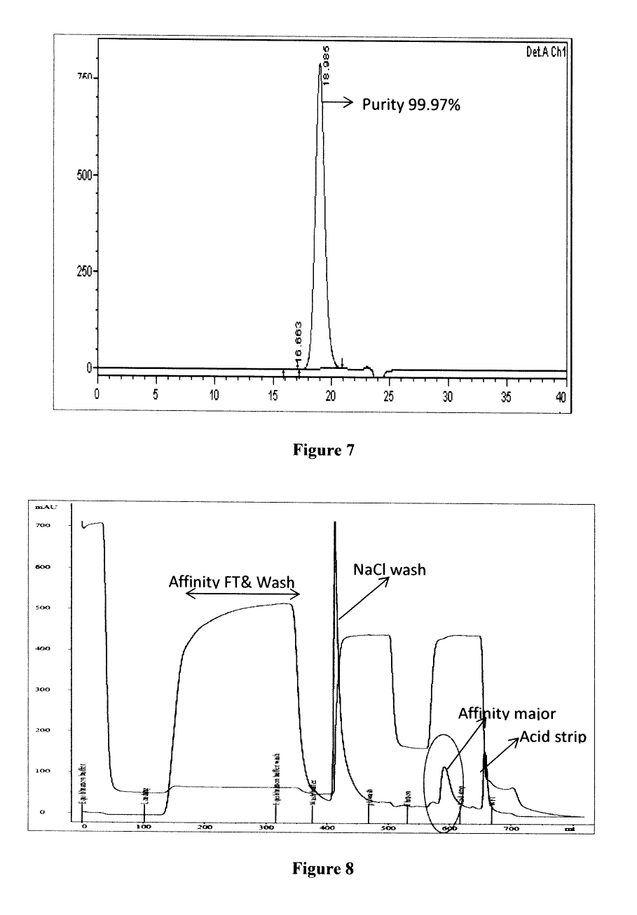

After final purification, purity of the recombinant FSH is observed to be at least 99%, as assessed by HP-SEC shown in FIG. 7.

After final purification, isoform profile of the purified recombinant FSH protein is observed to be similar to the standard.

### Example 2

### Purification of Urinary HCG

### Step 1: Affinity Column Chromatography

u-HCG crude mixture after reconditioning is passed through an affinity column matrix to capture the desired protein, selectively and to elute, thereafter. Prior to elution of the desired protein, the affinity matrix undergoes an intermediate column wash. The desired protein is eluted from the column at acidic pH. The column chromatography profile is shown in FIG. 8.

### Step 2: Ultrafiltration-Diafiltration and Reconditioning

The affinity column-eluted protein is reconditioned by UF/DF using 10 kDa MWCO membrane filter against low ionic strength buffer of pH 7.0 in order to match to the next column (Q column) step equilibration buffer conditions (e.g. pH and conductance). Diafiltered protein solution is passed through a 0.22 μm filter, prior to loading on to the Q-column.

### Step 3: Viral Inactivation

Diafiltered protein solution is incubated at the same pH condition in the presence of solvent/detergent or detergent for about 4-6 hours, under room temperature condition with constant stirring for viral inactivation.

### Step 4: Anion Exchange Column Chromatography (AEX)

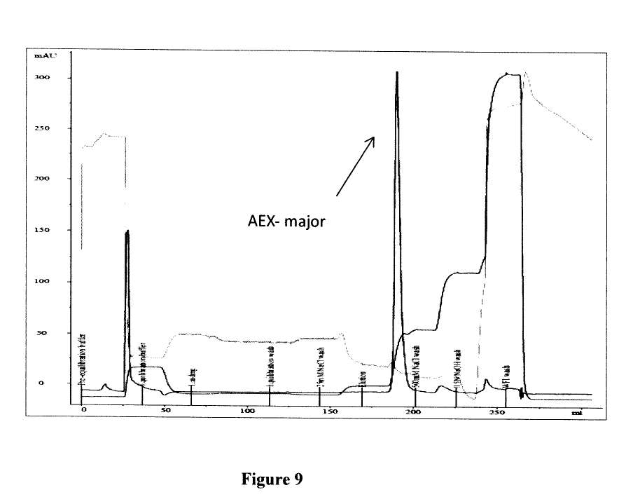

After diafiltration, solution containing u-HCG is loaded on to an anion exchange column for further purification of the desired protein with desired isoforms profile. This column step is carried out in bind-elute mode and is performed mainly for the removal of undesired isoforms of recombinant follicle stimulating hormone, while isolating the said protein with desired isoforms. The column chromatography profile is shown in FIG. 9. Protein is loaded on to the column at about pH 8.0 to bind to the matrix. Column matrix is washed with the same equilibration buffer to remove the unbound contaminants. Following the equilibration buffer wash, a second wash is performed with a buffer of pH lower than the initial equilibration buffer pH. Subsequently, a third wash is performed at acidic pH in the presence of NaCl. Column is re-equilibrated with the equilibration buffer and elution of the desired protein is carried out with an increase in conductance.

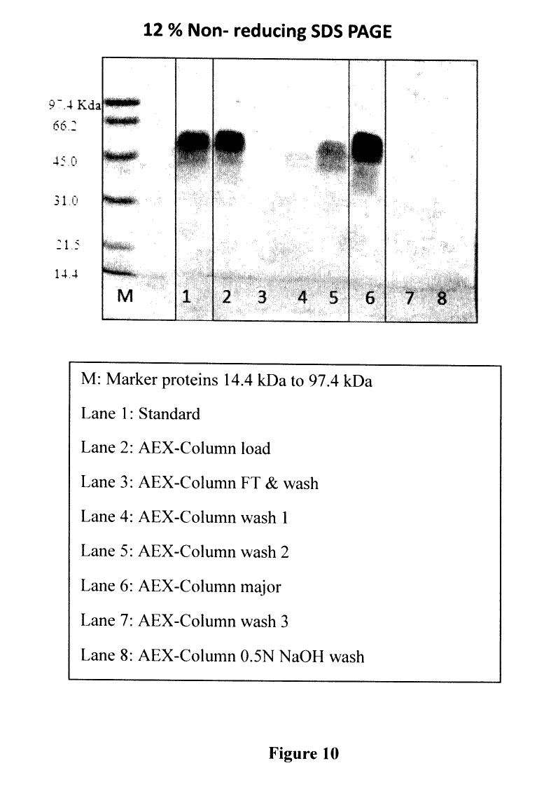

After the Q-column step, single band purity is observed by SDS PAGE, as shown in FIG. 10.

### Step 5: Ultrafiltration-Diafiltration

After the third column step, solution containing u-HCG undergoes an ultrafiltration-diafiltration step for buffer exchange, under room temperature conditions.

### Step 7: Microfiltration

Finally, the purified u-HCG solution is passed through a 0.22 μm membrane filter, aseptically, and is stored either in the liquid form under cold condition (for short-term storage) or under frozen condition for long-term storage at a concentration between 0.2 mg/mL and 2.5 mg/mL.

After final purification, isoform profile of the purified u-HCG is observed to be similar to the standard u-HCG.

### Example 3

### Purification of Urinary FSH

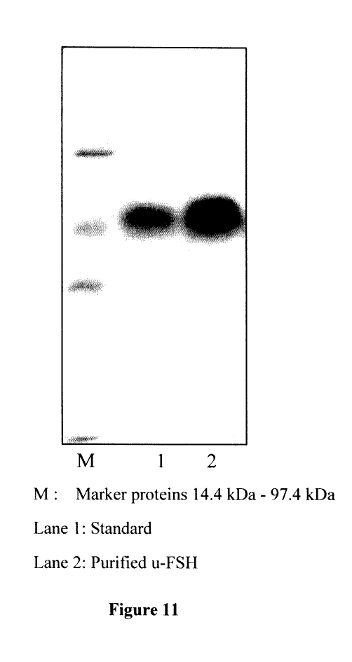

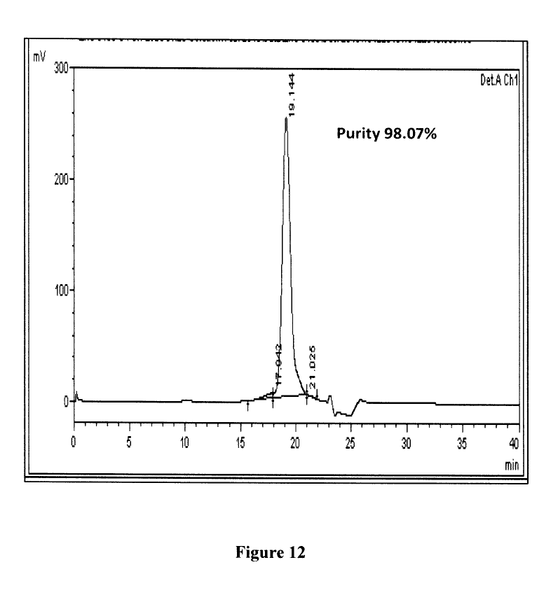

The purification process of u-FSH was carried out in the manner as described in the example 2. The purified u-FSH exhibits single band purity in gel, as assessed by SDS-PAGE (FIG. 11) and more than 98% purity, as assessed by HP-SEC (FIG. 12).

1. A process of purification of gonadotropin comprising the sequential steps of:
(a) affinity chromatography; and
(b) anion exchange chromatography, followed by any other purification steps.

2. The process as claimed in claim 1, wherein the affinity chromatography step is a gonadotropin-specific affinity matrix.

3. The process as claimed in claim 1, wherein elution of gonadotropin after step (a) is carried out at neutral buffer pH condition or under acidic pH condition.

4. The process as claimed in claim 1, wherein an anion exchanger for the step of anion exchange chromatography is selected from diethylaminoethyl functionalized agarose, mono quaternary ammonium functionalized agarose, and quaternary ammonium functionalized agarose.

5. The process of purification of gonadotropin as claimed in claim 1 comprising sequentially the following steps of:
(a) affinity chromatography;
(b) anion exchange chromatography; and
(c) hydrophobic interaction chromatography
wherein steps (b) and (c) can be carried out in any order.

6. The process as claimed in claim 5, wherein a hydrophobic column matrix is selected from phenyl sepharose, butyl sepharose, and octyl sepharose.

7. The process as claimed in claim 5, wherein in step (c) the gonadotropin is eluted from a column with down-the-gradient salt concentration.

8. The process as claimed in claim 7, wherein the salt is selected from ammonium sulphate, sodium chloride, ammonium chloride and sodium sulphate.

9. The process of purification of gonadotropin as claimed in claim 1 from a crude mixture comprising the steps of:
(a) cell separation and reconditioning;
(b) affinity column chromatography;
(c) ultrafiltration-diafiltration and reconditioning;
(d) viral inactivation;
(e) anion Exchange column Chromatography (AEX);
(f) reconditioning;
(g) hydrophobic interaction column chromatography (HIC);
(h) ultrafiltration-diafiltration;
(i) nanofiltration;
(j) microfiltration
wherein the hydrophobic and anion exchange chromatography steps can be performed in any order after the affinity chromatography steps; and wherein steps (c) to (j) can be carried out in any order.

10. The process as claimed in claim 9, wherein a diafiltration medium is selected from Tris-Cl, buffer, phosphate buffer, acetate buffer, citrate buffer, succinate buffer and combination thereof.

11. The process as claimed in claim 5, wherein anion exchange chromatography is carried out with an anion exchanger selected from diethylaminoethyl functionalized agarose, mono quaternary ammonium functionalized agarose, and quaternary ammonium functionalized agarose and hydrophobic interaction chromatography is carried out with a hydrophobic column matrix selected from phenyl sepharose, butyl sepharose, and octyl sepharose.

12. The process as claimed in claim 1, wherein the gonadotropin is either cell culture derived or crude mixture derived from urine.

13. The process as claimed in claim 1, wherein gonadotropin is selected from follicle stimulating hormone, luteinizing hormone, human chorionic gonadotropin and combination thereof.

14. The process as claimed in claim 1, wherein gonadotropin is selected from r-hFSH, u-FSH, r-hLH, u-LH, r-hHCG and u-HCG.

15. The process as claimed in claim 9, wherein anion exchange chromatography is carried out with an anion exchanger selected from diethylaminoethyl functionalized agarose, mono quaternary ammonium functionalized agarose, and quaternary ammonium functionalized agarose, and hydrophobic interaction chromatography is carried out with a hydrophobic column matrix selected from phenyl sepharose, butyl sepharose, and octyl sepharose.
